(19) United States
(12) Patent Application Publication
(10) Pub. No.: US 2007/0053851 A1
(43) Pub. Date: Mar. 8, 2007

(54) Orally effective, essentially water-free topical agent containing one or several oxidation-sensitive substances

(76) Inventors: Philippe Emmanuel Maillan, Eschentzwiller (FR); Cornelia Rütti, Dottikon (CH); Christine Saecker, Biberstein (CH); Jürgen Herbert Vollhardt, Ramlinsburg (CH); Horst Westenfelder, Neustadt a. d. W. (DE)

Correspondence Address: Stephen M Haracz;Bryan Cave, 1290 Avenue of the Americas, New York, NY, 10104, US

(21) Appl. No.: 10/571,160

(22) PCT Filed: Aug. 31, 2004
(86) PCT No.: PCT/EP04/09701

§ 371 Date: Nov. 6, 2006

(30) Foreign Application Priority Data
Sep. 9, 2003 (DE) 03019674.5

(51) Int. Cl. A61K 8/97; A61K 36/82

(52) U.S. Cl. 424/58; 424/729

(57) ABSTRACT

The present invention relates to an agent with oral activity on the form of a toothpaste, a mouthwash or a mouthwash concentrate containing one or more substances sensitive to oxidation, characterised in that the agent has a water content according to Karl Fischer of 7.5 wt.-% or less.

The present invention relates to a topical agent with oral activity which is selected from a toothpaste, a mouthwash and a mouthwash concentrate and which comprises one or more substances sensitive to oxidation, especially one or more polyphenols of green tea. Surprisingly, it has been found that the stability of such an agent can be improved significantly if the agent has a water content of not more than 7.5 wt.-% based on the total weight of the agent.

A number of diseases and disorders occur in the region of the oral cavity, for example dental caries, gingivitis, inflammatory processes such as, in particular, periodontosis and halitosis which is usually caused by volatile sulfur compounds in the oral cavity. These are a result of the metabolic conversion of sulfur-containing amino acids and proteins by oral micro-organisms. In addition, diseases resulting from oxydative stress conditions may occur in the biological tissue of the oral cavity. Again, these are often inflammations.

In order to prevent and treat such diseases and disorders agents especially designed for the topical administration in the oral cavity and to the teeth such as, in particular, dental cleansers such as toothpaste or tooth gel, sprays, mouth-rinses and mouthwashes are used.

A number of active ingredients for use in such agents are known. For example, WO 99/17735 discloses the use of certain chelates for treating halitosis, just to mention one example from the many publications dealing with active ingredients for the oral treatment of the oral cavity. Fluorine compounds are known active ingredients against caries. The use of plant extracts in dental care products and products for treating the oral cavity is also common; illustrative examples are WO 02/092028 or FR 2 791 893.

Products with green tea extracts are well known. Especially in East Asian popular medicine, green tea has a long tradition as a curative drug. Over a thousand years ago, Chen Zang, a Chinese pharmacist (Tang dynasty, 618 to 907) went on record with the statement that every drug works only against a certain disease, while tea is a drug against all diseases.

The main ingredients of green tea extract are polyphenols, so called catechins. The catechin present predominantly with regard to its quantity is (−)-epigallocatechin gallate. The green tea catechins are characterised by a very strong antioxidising effect. In addition, they have antimicrobial activity and also act as a deodorant against various unpleasant odours. This combination of activities makes them generally interesting for an application in dental and oral care, for example for the prevention or treatment of caries, gingivitis, periodontosis and halitosis. The antibacterial activity acts as a protection against caries and gingivitis (plaque formation), while the antioxidising effect prevents inflammatory processes or mitigates their progress. The deodorising properties reduce bad breath by binding malodorous components on the one hand and, on the other hand, by controlling the sulfur-generating bacteria in the oral cavity by enzyme inhibition due to their antimicrobial activity, thus preventing the development of halitosis.

The use of epigallocatechin gallate, a green tea polyphenol, in oral care products is disclosed in EP 0 067 476, for example.

WO 02/02096 discloses that the wellbeing of the entire body may be improved by administration of certain topical oral compositions. Epigallocatechin gallate is mentioned as one possible ingredient among many. The oral formulations of the citations are not especially limited either and include toffee, chewing gum, tooth powder, etc. Water is mentioned as one possible carrier of the oral formulations which generally amounts to about 5% to about 70%, preferably about 20% to about 50% of the agent. The value is based on the entire water content including the water bound to excipients and on all oral formulations. The overall water content for a toothpaste is not given. About 2% to about 45% are given as the value of the water added as a carrier. The agents disclosed as an example all contain more than 6.5% of water added as the carrier. Since all of the toothpastes also have a significant content of precipitated silica which has a considerable water content, the overall water content of these agents will invariably exceed 8%. Formulations with the active ingredient epigallocatechin gallate are not prepared in any of the examples. Agents with a low water content which include the active ingredient epigallocatechin gallate are not disclosed on the citation. No storage tests were conducted.

The use of green tea extracts containing polyphenol in topical agents with oral activity is difficult, because the polyphenols present in green tea are extremely reactive. They have a strong antioxidising effect which results in the formation of degradation products staining formulations containing such polyphenols brown upon extended storage. Even though only a small portion of the polyphenols is decomposed, this is usually sufficient to stain toothpastes and mouthwashes brown. Especially in dental care products and products for the treatment of the oral cavity, particularly in toothpaste formulations and mouthwashes, a brown discolouration is unacceptable and leads to the rejection of the product by the customer. As a result of these problems, extracts of green tea and green tea polyphenols have not been used practically in dental care products and products for the treatment of the oral cavity such as toothpastes or mouthwashes until today.

Other active ingredients, especially retinoids, ascorbyl compounds and ascorbic acid, resveratrol and flavonoids display similar problems in topical agents with oral activity, because these compounds are also subject to oxydative degradation. Again, stained degradation products or a deterioration of the effectiveness of the agents often result.

It is the object of the invention to provide novel topical agents with oral activity which, if possible, are capable of treating the entire spectrum of diseases and disorders occurring in teeth and the oral cavity and are able to both act against caries and gingivitis and reliably control halitosis or prevent the development thereof, which are active in case of inflammations of the oral cavity and especially periodontosis, and which may also be used to treat oxydative stress conditions in biological tissues of the oral cavity. The agents should have an attractive appearance to make them marketable as toothpaste and mouthwash concentrates or mouthwashes and keep this appearance even after lengthy storage at room temperature or at least undergo less of a change than products of the prior art.

The problem of the decomposition of substances sensitive to oxidation and especially the discolouration of green tea polyphenols usually does not occur in products such as chewing gum, candy, toffee and tablets, for example tablets for chewing or sucking, possibly because these products are coloured. However, it is found especially in toothpastes, mouthwashes and mouthwash concentrates.

The solution to this problem is based on the surprising finding that the stability of substances sensitive to oxidation and especially polyphenols of green tea may be improved considerably if these substances, especially the polyphenols, are formulated in agents having a water content of not more than 7.5 wt.-% based on the total weight of the agent. If these agents comprise additional components favouring the decomposition of substances sensitive to oxidation, especially green tea phenols, such as alkaline components like scrubbing agents on the basis of silica or calcium often contained in toothpastes, the stability of the substances sensitive to oxidation, especially green tea polyphenols may be further improved by conditioning these components with an acid.

The invention thus provides a topical agent with oral activity selected from toothpastes, mouthwashes and mouthwash concentrates comprising one or more substances sensitive to oxidation and especially green tea polyphenols having a water content of 7.5 wt.-% or less. The invention further relates to the use of substances sensitive to oxidation and especially green tea polyphenols for preparing such an agent having a water content of 7.5 wt.-% or less for the topical treatment of the oral cavity and/or the teeth. Finally, the invention relates to a method for decreasing the decomposition substances sensitive to oxidation and especially the dicolouration of green tea polyphenols in such a topical agent with oral activity which is characterised in that the water content of the agent is adjusted to 7.5 wt.-% or less.

According to the invention, it has also been found that water used for the preparation of the topical agents with oral activity and added to the mixture as a carrier (“free water”) affects the stability of the substances sensitive to oxidation and especially the green tea polyphenols much more severely than water present in a form bound to excipients and additives, such as water introduced into the agent via silica gel or titania.

The invention therefore also relates to a method for preparing a topical agent with oral activity containing substances sensitive to oxidation and especially green tea phenols selected from toothpastes, mouthwashes and mouthwash concentrates to which less than 2 wt.-% of water is added as free water not bound to excipients or additives and the oral agents obtainable by this method.

Unless stated otherwise or obvious, percentages always relate to the weight of a component based on the total weight of the agent (% w/w).

In the invention, a “topical agent with oral activity” is understood to mean an agent introduced into the oral cavity where it exerts a medical, hygienic or cosmetic effect on the teeth and/or other regions of the oral cavity. These agents according to the invention are toothpaste, mouthwashes and mouthwash concentrates. The term “oral cavity” means the entire region of the mouth and pharynges which is amenable to a topical treatment.

In German, a mouthwash may also be termed “mouth water”. However, the more specific term “mouthwash” is preferred, because the mouthwashes of the invention are essentially free of water. The mouth wash concentrates of the invention are generally formulated into finished mouthwashes before use by the end user by adding a diluent, generally water.

The substances sensitive to oxidation are not especially limited in the invention. In particular, these are retinoids, ascorbyl compounds, ascorbic acid, resveratrol, ubiquinones, flavonoids and/or green tea polyphenols. The flavonoids are preferred; green tea polyphenols are especially preferred. Ascorbyl compounds that may be mentioned are especially ascorbyl phosphates such as the ascorbyl phosphates sold under the trademark Stay-C, for example sodium ascorbyl phosphate (Stay-C50).

Conceptionally, the group of retinoids includes all retinoids that are not harmful for the use in oral agents including retinol and esters thereof, retinal as well as retinoic acid and esters thereof.

Retinol is characterised by the following structure:

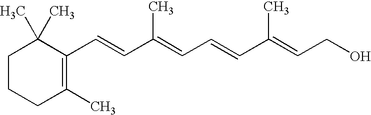

Retinol (also axerophthol; [3,7-dimethyl-9-(2,6,6-trimethyl-1-cyclohexenyl)-2,4,6,8-nonatetraene-1-ol] is synonymous to vitamin A₁; analogous to the derivatives retin-1-carboxylic acid (vitamin A acid, retinoic acid, tretinoin) and their esters or retin-1-al (vitamin A aldehyde) it is occasionally called vitamin A alcohol.

Preferably, the retinol esters of the invention have the structure 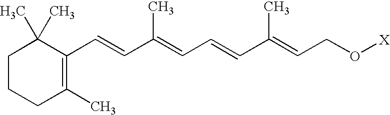
wherein X preferably is a branched or unbranched alkandyl or alkenoyl residue having 1 to 25 carbon atoms. Preferably, retinol palmitate (=retinyl palmitate) is selected as the retinol ester.

Retinal is characterised by the structure

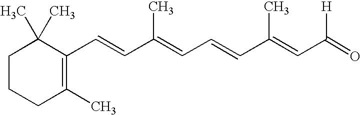

Retinal (vitamin A1 aldehyde, 3,7-dimethyl-9-(2,6,6-trimethyl-1-cyclohexenyl)-2,4,6,8-nonatetraenal] is most stable in its all-trans form. Retinal is generated by oxydative cleaving of carotene.

Retinoic acid [vitamin A acid, all-trans-3,7-dimethyl-9-(2,6,6-trimethyl-1-cyclohexenyl)-2,4,6,8-nonatetraenic acid] is characterised by the structure

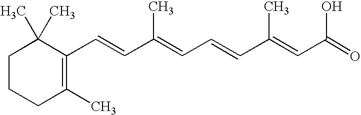

The ester group that may optionally be present instead of the hydrogen atom of the acid is preferably an alkyl group with 1 to 25 carbon atoms.

Not only the above trans-compounds, but also the analogous compounds having a cis-configuration at one or more double bonds may be used in the invention. If the above compounds are capable of displaying optical activity, both the individual enantiomers or diastereomers and any mixtures thereof, especially racemic mixtures, may be used in the invention.

In the invention, ascorbic acid is especially L-ascorbic acid {(R)-5-[(S)-1,2-dihydroxyethyl]-3,4-dihydroxy-5-H-furan-2-one, vitamin C} of the structural formula

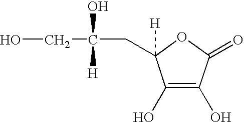

It is well soluble in water, well soluble in alcohol, insoluble in ether, petroleum ether, chloroform, benzene as well as in fats and fatty oils. Other stereochemical forms of ascorbic acid are also included.

Ascorbyl compounds preferably mean ascorbyl esters of fatty acids, especially preferably ascorbyl palmitate. Ascorbyl compounds within a narrower meaning are especially ascorbyl esters of the general structure 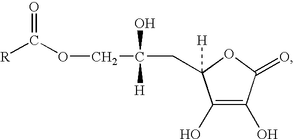
wherein R may be a branched or unbranched alkyl radical having up to 25 carbon atoms.

Ascorbyl glycosides are also included in the present invention, especially the ascorbyl glucosides, in particular ascorbyl-2-glucoside of the structure

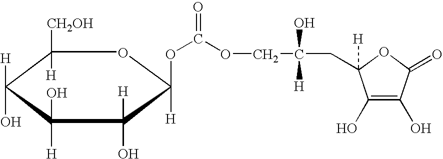

Ascorbyl compounds by a narrower definition are also ascorbyl phosphates, especially preferably the ascorbyl-2-phosphates of ascorbic acid and, respectively, the alkaline, alkaline earth and zinc salts thereof and mixed salts of such cations.

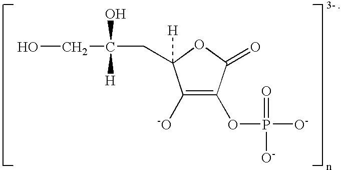

The above formula shows an ascorbyl phosphate ion with triple deprotonation even though other deprotonation stages are also advantageous within the meaning of the present invention.

The sodium, magnesium and zinc salts, for example sodium ascorbyl phosphate, are preferred.

Flavones or flavonoids are well known and are often used in cosmetic and dermatological applications. In this case, particular reference may be made to WO 00/74641 which may be perused for further details.

Flavone and its derivatives (often called “flavones” collectively) are characterised by the following basic structure (substitution position marked):

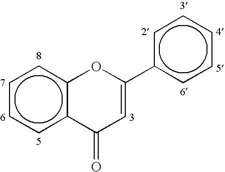

Some of the more important flavones which are also found in living nature are listed in the following table:

| | OH-substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually found in glycosidated form.

Other flavonoids that may be used in the invention are flavonones 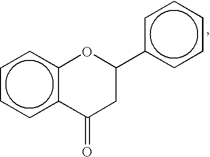
3-hydroxyflavones 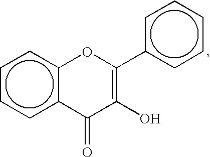
aurones 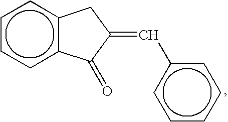
and isoflavones

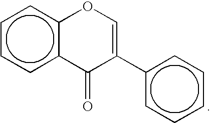

Substances sensitive to oxidation within the meaning of the invention are also flavonoids of the generic structural formula 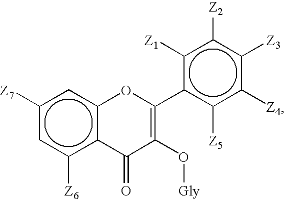
wherein Z₁ to Z₇ are independently selected from the group H, OH, alkoxy and hydroxy alkoxy, which alkoxy and hydroxy alkoxy groups may be branched or unbranched and have 1 to 18 carbon atoms, Gly being selected from the group of mono- and oligoglycoside radicals,
flavonoids of the generic structural formula 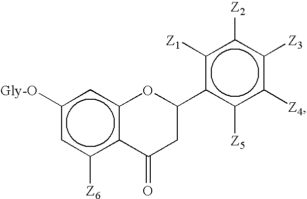
   wherein Z₁ to Z₆ are independently selected from the group H, OH, alkoxy and hydroxy alkoxy, which alkoxy and hydroxy alkoxy groups may be branched or unbranched and comprise 1 to 8 carbon atoms, Gly being selected from the group of mono- and oligoglycoside radicals,
flavonoids of the generic structural formula 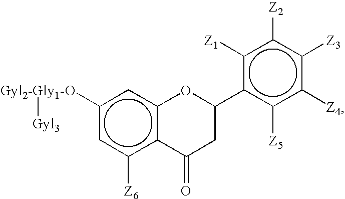
   wherein Z₁ Z₆ are as defined above and Gly₁, Gly₂ and Gly₃ are, independently, mono- or oligoglycoside radicals,
flavone glycosides of the structure 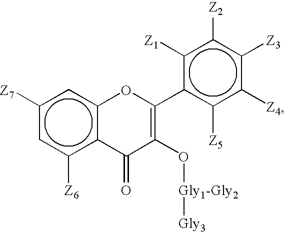
   e.g. of the structure 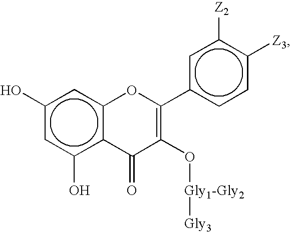
   wherein Z₁to Z₇, Gly₁, Gly₂ and Gly₃ are as defined above.

Gly₂ and Gly₃, respectively, may individually or jointly be saturations by hydrogen atoms.

It is preferred that Gly, Gly₁, Gly₂ and Gly₃ are independently selected from the group of hexosyl radicals, especially the rhamnosyl radicals and glucosyl radicals. However, other hexosyl radicals such as allosyl, altrosyl, galactosyl, gulosyl, idosyl, mannosyl and talosyl or pentosyl radicals may also be used.

It is preferred to independently select Z₁ to Z₇ from the group H, OH, methoxy, ethoxy and 2-hydroxyethoxy.

Special mention may be made of α-glucosylrutin, α-glucosylmyrictrin, α-glucosyl-isoquercitrin and α-glucosylquercitrin.

Exemplary flavonoids are α-glucosylrutin, naringin (aurantiin, naringenin-7-rhamnoglucosid), hesperidin (3′,5,7-trihydroxy-4′-methoxyflavanone-7-rutinoside, hesperidoside, hesperetin-7-O-rutinoside), rutin (3,3′,4′,5,7-pentahydroxyflavon-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)-flavon-3-(6-O-(6-deoxy-α-L-mannopyranosyl)-β-D-glucopyranoside)), monoxerutin (3,3′,4′5-tetrahydroxy-7-(2-hydroxyethoxy)-flavone-3-(6-O-(6-deoxy-α-L-mannopyranosyl)-β-D-glucopyranoside)), dihydrorobinetin (3,3′,4′,5′,7-pentahydroxyflavanon), taxifolin (3,3′,4′,5,7-pentahydroxyflavanon), eriodictyol-7-glucoside (3′,4′,5,7-tetrahydroxyflavanon-7-glucoside), flavanonmarein (3′,4′,7,8-tetrahydroxy-flavanon-7-glucoside) and isoquercitrin (3,3′,4′,5,7-pentahydroxyflavanon-3-(β-D-glucopyranoside).

Another substance sensitive to oxidation within the meaning of the invention is resveratrol, a stilbene derivative found in plants.

However, most preferably the substances sensitive to oxidation of the invention are one or more green tea polyphenols which display severe oxydative discolouration especially in toothpastes, mouthwashes and mouthwash concentrates of the prior art so that the products are commercially unattractive.

In the following, the invention will essentially be illustrated on the basis of the especially preferred green tea polyphenols, but the explanations apply analogously to the other substances sensitive to oxidation as described above.

When “green tea polyphenols” are mentioned within the framework of this invention, this means polyphenol compounds present in green tea which may be recovered from green tea in the customary manner by extraction and, optionally, other separation techniques such as especially the chromatographic separation technique. Of the polyphenols present in green tea, the compound (−)-epigallocatechin-3-gallate (EGCG) 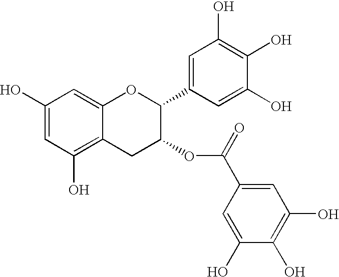
is the most important polyphenol compound which is also present in the largest quantity ratios. Other polyphenol compounds present in green tea are well known to the skilled practitioner. The following compounds may be mentioned:

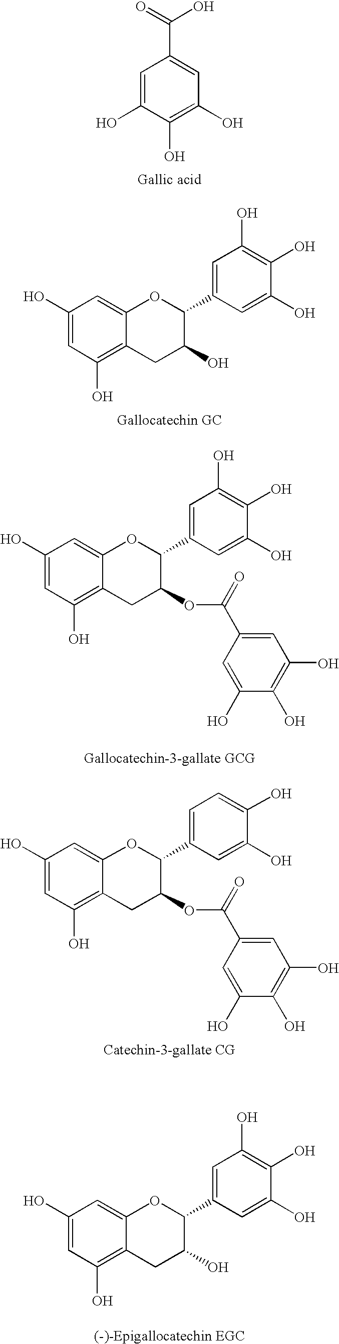

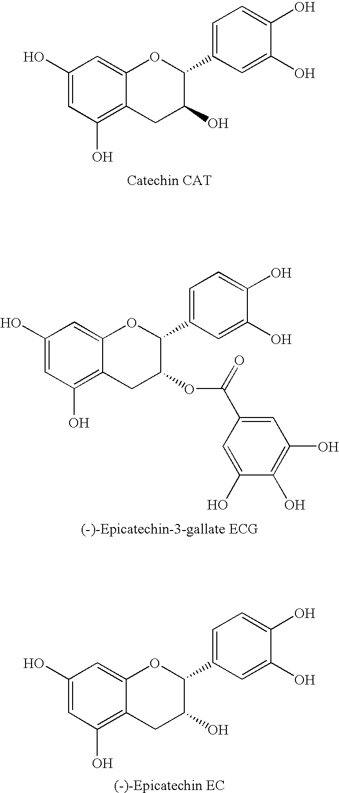

When the green tea polyphenols are optically active substances, a reference to such a substance is a reference to every single stereoisomer, especially each of the optically active enantiomers (preferably to the optically active enantiomer found in nature) and to any mixtures thereof, especially racemates.

According to the invention the term “green tea polyphenols” comprises all polyphenols present in green tea, especially the compounds mentioned above and most particularly the compound (−)epigalocatechin-3-gallate. The green tea polyphenols may be recovered from green tea in the usual manner, for example by extraction of the green tea leaves with hot water, thus obtaining an extract 40% of which consists of polyphenols and the typical ingredients which are given in table 1. This is based on the publication by Ballentine D. A. et al., 1997, The NetherlandsCrit Rev Food Sci Nutr. 1997 December; 37(8):693-704 which is included by reference.

| Ingredient | |
|---|---|
| | Polyphenol fraction |
| | (% of the dry substance) |
| Flavanols (comprising, e.g., EGCG) | 25.0 |
| Flavonols and flavonol glycoside | 3.0 |
| Phenolic acids and epsides | 5.0 |
| Other polyphenols | 3.0 |
| Caffeine | 3.0 |
| Theobromin | 0.2 |
| | Remaining ingredients |
| Amino acids | 4.0 |
| Organic acids | 0.5 |
| Monosaccharides | 4.0 |
| Polysaccharides | 13.0 |
| Cellulose | 7.0 |
| Protein | 15.0 |
| Lignin | 6.0 |
| Lipids | 3.0 |
| Chlorophyll and other pigments | 0.5 |
| Ash | 5.0 |
| Volatile ingredients | 0.1 |

The green tea polyphenols or flavones in the extract are composed as follows, for example:

| | mg/100 mg | |
|---|---|---|
| | dried tea leaves | ratio (%) |
| EGCG (Epigallocatechin gallate) | 13.37 | 73.99 |
| EGC (Epigallocatechin) | 0.44 | 2.43 |
| ECG (Epicatechin gallate) | 2.91 | 16.10 |
| EC (Epicatechin) | 0.55 | 3.04 |
| C (Catechin) | 0.02 | 0.11 |
| GCG (Gallocatechin gallate) | 0.26 | 1.44 |
| GA (Gallic acid) | 0.52 | 2.89 |

According to the invention, even an aqueous extract of green tea as described above, optionally after further concentration in order to reduce the water content, may be used to prepare the products of the invention. However, it is preferred to separate the remaining ingredients to the largest possible extent so that only the green tea polyphenols are used. It is preferred that the extract used, optionally after chromatographic purification, contain at least 30%, more preferably at least 50%, even more preferably at least 70%, especially at least 80%, for example at least 90%, most preferably at least 95% of green tea polyphenols, especially of the green tea polyphenols listed individually above, in each case given as % of the dry substance. Especially preferably, a product subjected to chromatographic purification is used, even more preferably a purified product with the highest possible increase of the epigallocatechin gallate content, for example up to 80% or more, preferably up to 90% or more based on the total amount of the green tea polyphenols, in each case given in % of the dry substance. Especially preferably, epigallocatechin gallate is used as green tea polyphenol in the invention. It is purified to the highest possible extent and contains only small traces (5% or less, preferably 3% or less, more preferably 2% or less) of the other green tea phenols, in each given as % of the dry substance.

The chromatographic purification of the aqueous green tea extract may be carried out in a manner known per se, as described, for example in “Separation of individual catechins from green tea using silica gel column chromatography and HPLC”, Journal of Food Lipids (2003), 10(2), 165-177. CODEN: JFFLES ISSN: 1065-7258. Reference may also be made to EP-A 1 077 211, EP-A 1 103 550, U.S. Pat. No. 6,210,679, WO-A 97/30597 and WO-A 95/18540 which disclose analogous work-ups of green tea extract and which are included by reference. Even though it is possible to use the aqueous green tea extract without chromatographic purification, said aqueous extract of green tea polyphenols often contains degradation products of the green tea polyphenols stained brown, which would give the agent according to the invention a faint brown discolouration. This may be acceptable in individual cases if the discolouration is not too strong, since such a discolouration does not increase significantly upon storage of the agent of the invention. However, this problem may be avoided altogether by using purified green tea extract and especially by using a polyphenol fraction separated by chromatography as described above and the topical agents with oral activity thus obtained have a particularly attractive colouring.

The amount of green tea polyphenols in the agent according to the invention is not particularly limited. As a rule, the content of substances sensitive to oxidation, especially green tea polyphenols, may be 0.01 wt.-% to 10 wt.-%, preferably 0.05 to 8 wt.-%, e.g. 0.1 to 5 wt.-%, in each case based on the total weight of the agent of the invention. A content of substances sensitive to oxidation, especially green tea polyphenols, but also of ascorbic acid and derivatives thereof, retinoids, especially retinol, but also retinyl derivatives such as retinyl palmitate, resveratrol and ubiquinon of 0.01 to 5 wt.-%, especially 0.01 to 3 wt.-%, e.g. 0.01 to 2 wt. % is also preferred.

Toothpaste, mouthwashes and mouthwash concentrates containing epigallocatechin-3-gallate in an amount of 0.01 to 10 wt.-%, more preferably 0.05 to 8 wt.-% and especially 0.1 to 5 wt.-%, in each case based on the total weight of the agent, are particularly preferred in the invention.

While a significant portion of water is generally employed in topical agents with oral activity, because water is an excellent carrier with physiological compatibility, it is essential for the invention that the agents do not contain more than 7.5 wt.-% of water based on the total weight of the agent, since an oxydative decomposition of the substances sensitive to oxidation or a discolouration of the green tea polyphenol will otherwise occur upon storage which results in an unacceptable brown discolouration of the agents, especially if these agents are toothpaste, mouthwash or mouthwash concentrates. The water content in toothpaste and mouthwashes or mouthwash concentrates is usually much higher. For example, DE-A 42 37 500 describes toothpastes having a water content of far above 30 wt.-%. WO 02/02096 also starts from a water content of 5 to 70 wt.-% in oral formulations and adds more than 6.5% as free water in the preparation of the agents disclosed as examples so that, taken together with the water bound to excipients and additives, a total water content of more than 8% invariably results.

The water content of the agents of the invention is preferably not more than 6.5 wt.-%, more preferably not more than 4.8 wt.-% and most preferably not more than 1.9 wt.-%, in each case based on the total weight of the agent. Unless otherwise indicated or obvious to the skilled practitioner, any water content given in the present application is the water content determined by the known method of Karl Fischer. The determination of the water content according to Karl Fischer is described, for example, in “Karl Fischer Titration: Determination of Water (Chemical Laboratory Practice/Anleitungen für die chemische Laboratoriums-praxis), Vol. 20, Eugene Scholz, Springer Verlag, 1984”.

As a result of the invention, it was surprisingly found that especially the water used as a carrier for the preparation of the agent of the invention (free water) affects the stability of the substances sensitive to oxidation and especially of the green tea polyphenols, while water bound to excipients and additives does not affect the stability as severely. When mixing the agents according to the invention, it is therefore preferred to use less than 2 wt.-% of free water, more preferably 1.5 wt.-% or less, even more preferably 1.0 wt.-% or less, more preferably 0.5 wt.-% or less. Most preferably, no free water at all is used. According to the invention, “free water” means water which is present as a liquid and is not bound adsorptively or as crystal water to solid substances such as silica gel, sorbitol or titania. The amount of free water in the agent according to the invention results from the amount of water used as a carrier during the preparation.

According to the invention, it was also found surprisingly that, in many cases, the stability of substances sensitive to oxidation, especially of green tea polyphenols, may be further increased in agents with oral activity if components of these agents that might affect the stability of the substances sensitive to oxidation, especially the green tea polyphenols are subjected to acidifying conditioning, i.e. treatment with an agent reacting in an acidic manner. If decomposition of the substances according to the invention, especially discolouration by decomposition of the green tea phenols, occurs despite the low water content of the agents of the invention, it may be determined by a few routine experiments which additives of the agent favour such decomposition. These additives may then be subjected to acidifying conditioning which will improve the compatibility of the additives and further reduce the instability of the agent of the invention, especially the discolouration caused by degradation of the green tea phenols. Such substances sensitive to oxidation which are particularly incompatible with green tea polyphenols but also with other substances sensitive to oxidation are often compounds reacting in a basic manner, i.e. compounds which generate a pH value of more than 7 in an aqueous suspension or solution.

Such alkaline additives are frequently used especially in toothpastes. Particularly the silica-based scrubbing agents often present in toothpastes may contribute to the decomposition of substances sensitive to oxidation, especially of the green tea polyphenols, to degradation products with a brown discolouration or a brown discolouration effect.

The decomposition of the substances sensitive to oxidation, especially the discolouration of the green tea polyphenols, is therefore reduced to an even greater degree in the invention if additives such a alkaline scrubbing agent, especially a silica-based scrubbing agent such as silica gel or a calcium-based agent which are often used in toothpastes but which are not compatible with substances sensitive to oxidation, especially the green tea polyphenols, are subjected to an acidifying conditioning step, i.e. a treatment with a compound reacting in an acidic manner (addition of the compound reacting in an acidic manner to the incompatible additive). The toothpastes of the invention preferably contain an alkaline scrubbing agent, especially a scrubbing agent on the basis of silica or aluminium dioxide such as silica gel or aluminium hydroxide, preferably in an amount of 10 to 30 wt.-%, more preferably 10 to 20 wt.-%.

“Compounds reacting in an acidic manner” in connection with the present invention are all compounds that provide a pH value of less than 7.0, more preferably a pH value of 6.0 or less, even more preferably a pH value of 5.0 or less, especially 4.0 or less in a 1% solution or dispersion. Suitable acidifying conditioning may be carried out with citric acid, for example 50% citric acid. Conditioning may be carried out either in a separate step, said alkaline compound, for example the silica-based scrubbing agent, being treated in a first step with the compound reacting in an acidic manner and then added to the agent according to the invention. Alternatively, it may be carried out during formulation of the agent of the invention. This is preferred and explained in the examples. Therefore, the agents of the invention preferably contain a compound reacting in an acidic manner, e.g. citric acid.

The compound reacting in an acidic manner is preferably present in an amount of 0.05 to 5%, especially 0.1 to 3%, in the agent of the invention, especially in the toothpaste of the invention.

The topical agents with oral activity of the invention are, for example, toothpastes which are the centre and base of any dental care and therefore often contain substances sensitive to oxidation or which, respectively, are the platform most suitable for the application of the multi-active effects of green tea phenols. Another important product form in dental care are mouthwashes. In addition to an antimicrobial effect they are especially popular because of their deodorising action. Green tea polyphenols once again have a double effect here. Therefore, the topical agents with oral activity of the invention are toothpastes, mouthwashes or mouthwash concentrates.

The topical agents with oral activity of the invention may be drugs, cosmetic products or hygiene products. The customary components and additives of toothpastes, mouthwashes and mouthwash concentrates of the invention are known to the skilled practitioner and described in pertinent standard publications such as “Formulierungstechnik” (formulation techniques), H. Mollet, A. Grubenmann, Wiley VCH 2000. The agents of the invention may especially contain additional active ingredients such as antibacterial active ingredients, for example additional extracts of natural products with antibacterial activity. Other possible additives to the agents of the invention are the usual anti-caries agents such as phosphates, pyrophosphates, polyphosphates, phosphonates, polyphosphonates and mixtures thereof. Polyacrylates or polycarboxylates, polyepoxysuccinates, ethylene diamine tetraacetic acid, nitrilotriacetic acid and similar compounds, agents for the prevention of odontolith or dental plaque, agents capable of releasing fluoride ions such as sodium fluoride, potassium fluoride, tin fluoride, ammonium fluoride and mixtures thereof, desensitising agents and painkillers such as strontium chloride, potassium nitrate, extracts of active ingredients of natural products and non-steroidal anti-inflammatory agents, agents to control bad breath such as the usual antimicrobial agents such as triclosan, phthalic acid and phthalic acid salts, chlorohexidin, hexetidin, sanguinarin, benzalkonium chloride, salicyl anilide, domiphen bromide, cetylpyridinium chloride, N-tetradecylpyridinium chloride, tetradecyl-4-ethylpyridinium chloride, octenifin, delmopinol, octapenol and other piperidin derivatives, nicin preparations, agents releasing zinc or tin ions, antibiotics such as augmentin, amoxicillin, tetracyclin, doxycyclin, minocyclin and metronidazol and methyl salicylates as well as metal cations and mixtures thereof generally used for this purpose may also be used for preparing the agents of the invention. A good survey of the customary additives in topical agents with oral activity may be found in WO 02/092028, for example, which is hereby included by reference.

The toothpastes of the invention may also contain the usual polishing materials (or a scrubbing agent) such as silica, aluminium dioxide, phosphates and mixtures thereof such as, for example, dicalcium orthophosphate dihydrate, calcium pyrophosphate, tricalcium phosphate, aluminium hydroxide, silica gel, etc. Calcium may also be included as a scrubbing agent; in practical applications, especially ground or precipitated calcium is used. Polyethylene fine powder may also be used as a scrubbing agent, e.g. fine powder with a particle size of <10 μm as distributed, for example, by BASF, Ludwigshafen, Germany. Toothpastes may also contain other customary additives, for example anionic, amphoteric, zwitterionic, cationic or non-ionic surfactants or mixtures thereof, antioxidants, agents for changing the colour of the teeth, especially agents for whitening the teeth, minerals, vitamins and nutrients, sweeteners, humectants, fillers, thickeners, alkali metal bicarbonates, buffers, colorants, flavouring agents, etc. Again, reference is made to WO 92/092028 in this respect.

In addition, the agents of the invention will contain at least one carrier material. A preferred carrier for the toothpastes especially preferred in accordance with the invention is glycerin, but other non-aqueous carriers known in the prior art such as propylene glycol and polyglycerin may also be used. The mouthwash concentrates or mouthwashes, respectively, may be prepared on the basis of alcohol (ethanol) or without alcohol. Mouthwash concentrates without alcohol have the advantage that they may be used by children without any risk. Mouthwash concentrates containing alcohol may, for example, be prepared on the basis of a glycerin ethanol mixture; a suitable carrier material for mouthwash concentrates without alcohol is glycerin, propylene glycol and polyglycerin.

The preparation of the products of the invention is carried out in a manner known per se analogously to methods known in the prior art as, for example, described in WO 02/092028 which is included by reference.

The following examples illustrate the invention.

### EXAMPLE 1

A toothpaste was prepared from the following ingredients
(all amounts in % w/w):

| A) | Glycerin (anhydrous) | ad 100 |
|---|---|---|
| | Epigallocatechin gallate (EGCG) | 0.10 |
| | Texapon N70 (sodiumlauryl sulfate) | 1.42 |
| | Sodium fluoride | 0.22 |
| | Sodium saccharin | 0.10 |
| | Carbopol 981 (carbomer) | 0.70 |
| | Syloblanc 81 (silica gel) | 10.00 |
| | Syloblanc 82 (silica gel) | 10.00 |
| B) | Citric acid | 0.45 |
| | Water (Aqua) | 0.30 |
| | | 100.00 |

by first mixing all of the substances listed under A in the order indicated at room temperature with slow stirring. Stirring was continued until a homogenous paste was obtained. The materials listed under B) were mixed in a separate vessel with slow stirring at room temperature and added to the homogenous paste at room temperature. Mixing was resumed until a homogenous paste was obtained. Said homogenous paste is suitable as a toothpaste and contains 0.1 wt.-% of epigallocatechin gallate. The total water content according to Karl Fischer was 4.7%.

### COMPARATIVE EXAMPLE 1

For comparison, a toothpaste containing 0.1 wt.-% of
epigallocatechin gallate and having the following composition
(amounts in % w/w) was prepared:

| Water | ad 100 |
|---|---|
| Glycerin | 20.00 |
| Epigallocatechin gallate (EGCG) | 0.10 |
| Texapon N70 (sodiumlauryl sulfate) | 1.42 |
| Sodium fluoride | 0.22 |
| Sodium saccharin | 0.10 |
| Carbopol 981 (Carbomer) | 1.40 |
| Syloblanc 81 (silica gel) | 10.00 |
| Syloblanc 82 (silica gel) | 10.00 |
| | 100.00 |

by first mixing all of the substances listed under A in the order indicated at room temperature with slow stirring. Stirring was continued until a homogenous paste was obtained. The total water content according to Karl Fischer was 59%.

### TEST EXAMPLE 1

The two toothpastes from example 1 and comparative example 1 were stored at room temperature (25° C.) for eight weeks and, independently, at 5° C. for eight weeks. Then the colour was evaluated by colorimetry. The calorimetric evaluation was carried out in accordance with a CIE 1976 L×a×b “Colorspace” evaluation as described in “Colour Physics for Industry” by Roderick McDonald, Society of Dyers & Colourists, March 1997. In accordance with ΔE=[(L−Lₒ)²+(a−aₒ)²+(b−bₒ)^{2/1} the value ΔE results from a so-called colour space, wherein L represents brightness, positive values for “a” mean red colours, negative values for “a” mean green colours, positive values for “b” mean yellow colours and negative values for “b” mean blue colours. The colour measurements were carried out with a Minolta Spectrophotometer CM-3600d.

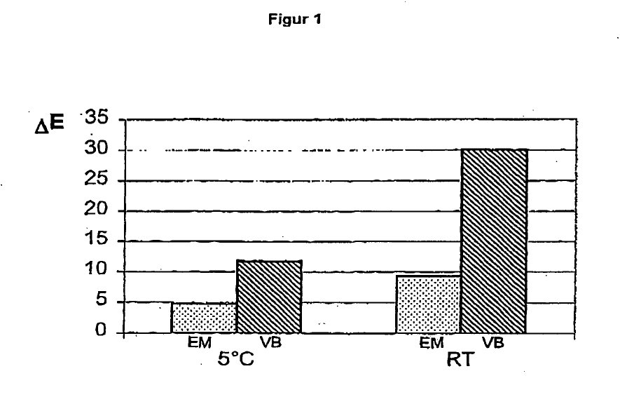

The result of the comparison is shown in FIG. 1. It is shown that, even at 5° C., the toothpaste according to the invention is discoloured to a much smaller degree than the water-based comparative product. The difference becomes especially evident at room temperature. Even at room temperature, the toothpaste of the invention is more stable than the comparative product after storage at only 5° C. The water-based toothpaste of the comparative example was so discoloured after eight weeks of storage at room temperature that the toothpaste is no longer suitable for commercial purposes. In contrast, the toothpaste of the invention showed only a slight discolouration which does not interfere with its commercial exploitation.

VB in FIG. 1 represents the toothpaste of the comparative example and EM is the toothpaste of the invention. ΔE indicates the “Colorspace” evaluation.

### EXAMPLE 2

A mouthwash concentrate was prepared from the following
ingredients (all amounts in % w/w)

| A) | Glycerin | ad 100 |
|---|---|---|
| | Sodium saccharin | 1.80 |
| B) | Epigallocatechin gallate (EGCG) | 2.50 |
| | Alcohol (ethanol) | 62.50 |
| | Propylene glycol | 5.00 |
| | | 100.00 |

by mixing sodium saccharin with glycerin at room temperature with slow agitation. In a separate vessel, epigallocatechin gallate was dissolved in ethanol at room temperature. The propylene glycol was added with stirring at room temperature. The mixture of the components B) was added to the mixture of the components A). A mouthwash concentrate on alcohol/glycerin basis with 2.5% of epigallocatechin gallate was obtained. The total water content according to Karl Fischer was less than 0.5%.

### COMPARATIVE EXAMPLE 2

A comparative mouthwash concentrate which contains water as
customary in the prior art and comprises the ingredients
(amounts in % w/w)

| A) | Water | ad 100 |
|---|---|---|
| | Glycerin | 5.00 |
| | Sodium saccharin | 1.80 |
| B) | Epigallocatechin gallate (EGCG) | 2.50 |
| | Alcohol (Ethanol) | 62.50 |
| | | 100.00 |

was prepared by dissolving sodium saccharin in water at room temperature and then adding glycerin. In a separate vessel, epigallocatechin gallate was dissolved in ethanol at room temperature, and the components b) were mixed with the components A). The total water content according to Karl Fischer was 29%.

### TEST EXAMPLE 2

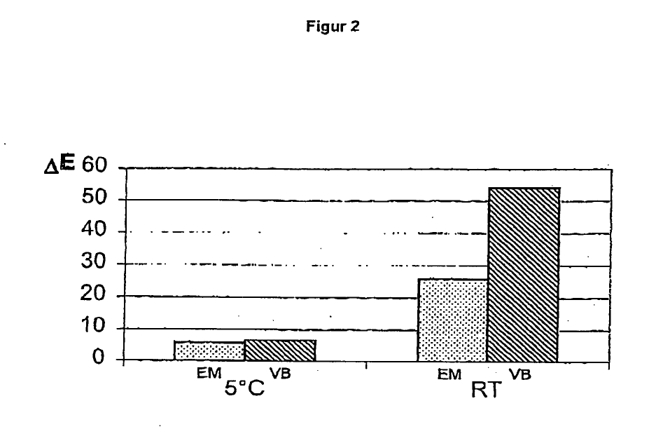

The mouthwash concentrates from example 2 and comparative example 2 were stored at room temperature for three months and, independently, at 5° C. for three months. Then a calorimetric determination as described in test example 1 was carried out; the result is shown in FIG. 2. While the discolouration of the mouthwash concentrate is still acceptable even for the comparative product at 5° C., the discolouration of the comparative product after three months of storage at room temperature is still so high that the product is unsuitable for commercial use. A certain discolouration is also found in the mouthwash of the invention which, however, is considerably lower than that of the comparative product.

In FIG. 2, VB represents the mouthwash concentrate of the comparative example and EM the mouthwash concentrate of example 2 of the invention. ΔE indicates the “Colorspace” evaluation.

### EXAMPLE 3

A mouthwash concentrate with the following ingredients
(amounts in % w/w)

| A) | Glycerin | ad 100 |
|---|---|---|
| | Sodium saccharin | 1.80 |
| B) | Epigallocatechin gallate (EGCG) | 2.50 |
| C) | Perfume oil (Peppermint 450-150 Dullberg) | 2.50 |
| | Propylene glycol | 30.00 |
| | | 100.00 |

was prepared by mixing sodium saccharin and glycerin at room temperature with slow agitation. Then epigallocatechin gallate was added and stirred until the epigallocatechin gallate was completely dissolved. In a separate vessel, the components C) were mixed at room temperature and then added to the mixture of A) and B). A mouthwash concentrate without alcohol containing 2.5% of epigallocatechin gallate was obtained. The total water content according to Karl Fischer was less than 0.5%.

### EXAMPLES 4 TO 17

Toothpastes were prepared in the manner known per se from the components listed in the following tables. Said toothpastes were stored at different temperatures and the content of the substance sensitive to oxidation determined in the customary manner after two weeks and three weeks. The results are also listed in the following tables. The total water content according to Karl Fischer in all toothpastes was less than 0.5%.

4. Toothpaste with Ascorbic acid

| Glycerin (anhydrous) | Ad 100 |
|---|---|
| Syloblanc 81 | 10.00 |
| Syloblanc 82 | 10.00 |
| Sodium-mono-fluorophosphate | 0.85 |
| Carboxymethyl cellulose | 1.20 |
| Aerosil 200 | 2.00 |
| Texapon K 12 powder | 1.20 |
| Ascorbic acid | 1.00 |
| Peppermint flavour | 1.00 |
| Titanium dioxides E 171 | 0.50 |
| Saccharin Na. | 0.20 |
| Cyclamate | 0.05 |

Vitamin C content [%]

| | |
|---|---|
| 2 weeks | 3 months |

| RT | 43° C. | 5° C. | RT | 43° C. |
|---|---|---|---|---|
| 1.00 | 1.00 | 1.00 | 1.00 | 0.95 |

5. Toothpaste with sodium ascorbyl phosphate (Stay-C50)

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Carboxymethyl cellulose | 1.20 |
| Aerosil 200 | 2.00 |
| Texapon K 12 powder | 1.20 |
| Stay-C 50 | 0.50 |
| Peppermint flavour | 1.00 |
| Titanium dioxides E 171 | 0.50 |
| Saccharin Na. | 0.20 |
| Cyclamate | 0.05 |

Stay-C50 content [%]

| | |
|---|---|
| 2 weeks | 3 months |

| RT | 43° C. | 5° C. | RT | 43° C. |
|---|---|---|---|---|
| 0.50 | 0.50 | 0.50 | 0.50 | 0.49 |

6. Toothpaste with Retinol

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Retinol 10S BASF | 1.00 |
| Peppermint flavour | 1.20 |
| Titanium dioxide E 171 | 0.50 |
| Saccharin Na. | 0.20 |
| Cyclamate | 0.05 |

Retinol content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.098 |

7. Toothpaste with retinyl palmitate

| Glycerin, anhydrous | Ad 100 |
|---|---|
| Syloblanc 81 | 10.00 |
| Syloblanc 82 | 10.00 |
| Sodium fluoride | 0.85 |
| Hydroxypropyl methylcellulose | 1.00 |
| Aerosil 200 | 2.00 |
| Texapon K 12 powder | 1.20 |
| Vitamin A Palmitat 1,7 mn. | 0.10 |
| Peppermint flavour | 1.20 |
| Titanium dioxide E 171 | 0.50 |
| Saccharin Na. | 0.20 |
| Cyclamate | 0.05 |

Retinyl palmitate content [%]

| | |
|---|---|
| 2 weeks | 3 months |

| RT | 43° C. | 5° C. | RT | 43° C. |
|---|---|---|---|---|
| 1.00 | 1.00 | 1.00 | 1.00 | 0.95 |

5. Toothpaste with Resveratrol

| Glycerin, anhydrous | Ad 100 |
|---|---|
| Precipitated chalk, shepherd's chalk | 40.00 |
| Sodium-mono-fluorophosphate | 0.85 |
| Hydroxypropylmethyl cellulose | 1.20 |
| Aerosil 200 | 2.00 |
| Texapon K 12 powder | 1.20 |
| Resveratrol | 0.10 |
| Aroma | 1.20 |
| Titanium dioxide E 171 | 0.50 |
| Saccharin Na. | 0.20 |
| Cyclamate | 0.05 |

Resveratrol content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.09 |

9. Toothpaste for the prevention of plaques

| Glycerin, anhydrous | Ad 100 |
|---|---|
| Zeodent 113 | 20.00 |
| Sodium-mono-fluorophosphate | 0.85 |
| Cellulose Gum | 0.80 |
| Aerosil 200 | 2.00 |
| Texapon K 12 powder | 1.20 |
| Resveratrol | 0.10 |
| Aroma | 1.20 |
| Titanium dioxide E 171 | 0.50 |
| Tetrapotassium pyrophosphate | 1.20 |

Resveratrol content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.094 |

10. Toothpaste with flavone

| Glycerin, anhydrous | Ad 100 |
|---|---|
| Precipitated chalk, shepherd's chalk | 40.00 |
| Sodium-mono-fluorophosphate | 0.85 |
| Hydroxypropylmethyl cellulose | 1.20 |
| Aerosil 200 | 2.00 |
| Texapon K 12 powder | 1.20 |
| Flavone | 0.10 |
| Aroma | 1.20 |
| Titanium dioxide E 171 | 0.50 |
| Saccharin Na. | 0.20 |
| Cyclamate | 0.05 |

Flavone content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.098 |

11. Toothpaste with Flavonol

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Flavonol | 0.10 |
| Peppermint flavour | 1.20 |
| Titanium dioxide E 171 | 0.50 |
| Saccharin Na. | 0.20 |
| Cyclamate | 0.05 |

Flavonol content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.09 |

12. Toothpaste with chrisin

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Chrisin | 0.10 |
| Peppermint flavour | 1.20 |
| Titanium dioxide E 171 | 0.50 |
| Saccharin Na. | 0.20 |
| Cyclamate | 0.05 |

Chrisin content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.094 |

13. Toothpaste with galangin

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Galangin | 0.10 |
| Peppermint flavour | 1.20 |
| Titanium dioxide E 171 | 0.50 |
| Saccharin Na. | 0.20 |
| Cyclamate | 0.05 |

Galangin content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.095 |

14. Toothpaste with apigenin

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Apigenin | 0.20 |
| Peppermint flavour | 1.20 |
| Titanium dioxide E 171 | 0.50 |
| Saccharin Na. | 0.20 |
| Cyclamate | 0.05 |

Apigenin content [%]

| | |
|---|---|
| 2 weeks | 3 months |

| RT | 43° C. | 5° C. | RT | 43° C. |
|---|---|---|---|---|
| 0.20 | 0.20 | 0.20 | 0.20 | 0.19 |

15. Toothpaste with Fisetin

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Fisetin | 0.10 |
| Peppermint flavour | 1.20 |
| Titanium dioxide E 171 | 0.50 |
| Saccharin Na. | 0.20 |
| Cyclamate | 0.05 |

Fisetin content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.90 |

16. Toothpaste with Quercetin

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Quercetin | 0.10 |
| Peppermint flavour | 1.20 |
| Titanium dioxide E 171 | 0.50 |
| Saccharin Na. | 0.20 |
| Cyclamate | 0.05 |

Quercetin content [%]

| | |
|---|---|
| 2 weeks | 3 months |

| RT | 43° C. | 5° C. | RT | 43° C. |
|---|---|---|---|---|
| 0.10 | 0.10 | 0.10 | 0.10 | 0.094 |

17. Toothpaste with ubiquinone

| Glycerin, anhydrous | Ad 100 |
|---|---|
| Syloblanc 81 | 10.00 |
| Syloblanc 82 | 10.00 |
| Sodium-mono-fluorophosphate | 0.85 |
| Hydroxypropylmethyl cellulose | 1.00 |
| Aerosil 200 | 2.00 |
| Texapon K 12 powder | 1.20 |
| Q 10 | 0.20 |
| Peppermint flavour | 1.20 |
| Titanium dioxide E 171 | 0.50 |
| Saccharin Na. | 0.20 |
| Cyclamate | 0.05 |

Q 10 content [%]

| | |
|---|---|
| 2 weeks | 3 months |

| RT | 43° C. | 5° C. | RT | 43° C. |
|---|---|---|---|---|
| 0.20 | 0.20 | 0.20 | 0.20 | 0.192 |

1. A topical agent with oral activity in the form of a toothpaste, a mouthwash or a mouthwash concentrate comprising one or more green tea polyphenols, characterized in that the agent has a water content according to Karl Fischer of 7.5 wt.-% or less.

2. A topical agent with oral activity according to claim 1, characterized in that it contains one or more green tea polyphenols in an amount of 0.01 to 10 wt.-% based on the weight of the agent.

3. A topical agent with oral activity according to claim 1, characterized in that it contains epigallocatechin gallate.

4. A topical agent with oral activity according to claim 1, characterized in that it comprises an extract of green tea.

5. A topical agent with oral activity according to claim 1, characterized in that the agent has a water content according to Karl Fischer of 4.8 wt.-% or less.

6. A topical agent with oral activity according to claim 5, characterized in that it has a water content according to Karl Fischer of 3 wt.-% or less.

7. A topical agent with oral activity according to claim 1, characterized in that the agent is a toothpaste.

8. A topical agent with oral activity according to claim 7, characterized in that the toothpaste has a scrubbing constituent reacting in a basic manner, preferably a scrubbing constituent on the basis of silica or calcium.

9. A topical agent with oral activity according to claim 8, characterized in that the toothpaste contains polyethylene fine powder as the scrubbing constituent.

10. A topical agent with oral activity according to claim 1, characterized in that the agent is a mouthwash or a mouthwash concentrate.

11. A topical agent with oral activity according to claim 1, characterized in that it further contains a compound reacting in an acidic manner.

12. A topical agent with oral activity according to claim 11, characterized in that the compound reacting in an acidic manner is citric acid.

13. A topical agent with oral activity according to claim 1, characterized in that less than 2 wt.-% of free water were added to it.

14. A method for making a topical agent in the form of a toothpaste, a mouthwash or a mouthwash concentrate comprising mixing a green tea polyphenol with a carrier and adjusting and/or maintaining the water content of the topical agent according to Karl Fischer of to 7.5 wt.-% or less.

15. A method according to claim 14, characterized in that the agent is intended for the prevention and/or treatment of caries, gingivitis and/or halitosis.

16. A method according to claim 14, characterized in that the agent is intended for the prevention and/or the treatment of inflammatory processes in the oral cavity.

17. A method according to claim 16, characterized in that the inflammatory processes in the oral cavity are periodontosis.

18. A method according to claim 14, characterized in that the agent is an agent for the prevention and/or treatment of oxidative stress conditions in the biological tissue of the oral cavity.

19. A method for reducing the discoloration of green tea polyphenols in a topical agent with oral activity selected from toothpaste, mouth wash or mouthwash concentrate, comprising adjusting the water content of the agent according to Karl Fischer to 7.5 wt.-% or less.

20. (canceled)

21. A method for preparing a topical agent with oral activity selected from toothpaste, mouthwash and mouthwash concentrate comprising mixing one or more green tea polyphenols with a carrier or additive, wherein the carrier or additive comprises less than 2 wt.-% of water.

22. A method according to claim 21, wherein the carrier or additive comprises 1.5 wt.-% of water or less.

23. A topical agent with oral activity, selected from the group consisting of toothpaste, mouthwash and mouthwash concentrate produced by the method according to claim 21.

24. A method according to claim 21, wherein the green tea polyphenols are formulated with a compound reacting in an acidic manner.
